(19) United States
(12) Patent Application Publication
(10) Pub. No.: US 2012/0048270 A1
(43) Pub. Date: Mar. 1, 2012

(54) POWDER INHALER DEVICE

(75) Inventor: Jean-Marc Pardonge, Le Authieux Sur Port Saint Ouen (FR)

(73) Assignee: VALOIS SAS, Le Neubourg (FR)

(21) Appl. No.: 13/319,654

(22) PCT Filed: Jun. 8, 2010
(86) PCT No.: PCT/FR10/51130

§ 371 Date: Nov. 9, 2011

(30) Foreign Application Priority Data
Jun. 11, 2009 (FR) FR 09 53883

(51) Int. Cl. A61M 15/00

(52) U.S. Cl. 128/203.15

(57) ABSTRACT

A powder inhaler device having a body provided with a dispenser orifice; a reservoir containing a dose of powder to be dispensed; a reservoir opening mechanism for opening a reservoir on each actuation; a dispersion chamber including an outlet that is connected to the dispenser orifice, and an inlet that is connected to the opening mechanism and that receives the dose of powder from the open reservoir. The dispersion chamber containing a movable element, such as a ball. The device further having a remote drive mechanism for remotely driving the movable element, the drive mechanism acting without contact with the movable element so as to set it into motion in the dispersion chamber.

The present invention relates to a powder inhaler device, and more particularly to a dry-powder inhaler.

Inhalers are well known in the prior art. Various types exist. A first type of inhaler contains a reservoir receiving many doses of powder, the inhaler being provided with metering means making it possible, on each actuation, to remove one dose of said powder from the reservoir, so as to bring said dose into an expulsion duct in order to be dispensed to the user. Inhalers including individual reservoirs, such as capsules, that are loaded into the inhaler just before said reservoir is used are also described in the prior art. The advantage of such devices is that it is not necessary to store all of the doses inside the appliance, such that said appliance can be compact. However, the inhaler is more difficult to use, since the user is obliged to load a capsule into the inhaler before each use. Another type of inhaler consists in packaging the doses of powder in individual predosed reservoirs, then in opening one of the reservoirs each time the inhaler is actuated. That implementation seals the powder more effectively since each dose is opened only when it is about to be expelled. In order to make such individual reservoirs, various techniques have already been proposed, such as an elongate blister strip or blisters disposed on a rotary circular disk. All existing types of inhalers, including those described above, present both advantages and drawbacks associated with their structures and with their types of operation. Thus, with certain inhalers, there is the problem of metering accuracy and reproducibility on each actuation. In addition, the effectiveness of the dispensing, i.e. the fraction of the dose that effectively penetrates into the user's lungs in order to have a beneficial therapeutic effect, is also a problem that exists with a certain number of inhalers. A solution for solving that specific problem has been to synchronize the expulsion of the dose with the inhalation of the patient. Once again, that can create drawbacks, in particular in that type of device, the dose is generally loaded into an expulsion duct before inhalation, then expulsion is synchronized with inhalation. That means that if the user drops, shakes, or manipulates the inhaler in an undesirable or inappropriate manner between the moment when the user loads the dose (either from a multidose reservoir or from an individual reservoir) and the moment when the user inhales, then the user risks losing all or part of the dose, with said dose possibly being spread about inside the appliance. In that event, there can exist a high risk of overdosing the next time the device is used. The user who realizes that the dose is not complete will load a new dose into the appliance, and while the new dose is being inhaled, a fraction of the previous dose that was lost in the appliance could thus be expelled at the same time as the new dose, thereby causing an overdose. In the treatments envisaged, such overdosing can be very harmful, and the authorities in all countries are issuing ever-stricter requirements to limit the risk of overdosing as much as possible. With regard to opening the individual reservoirs, it has been proposed to peel off or to unstick the closure layer. That presents the drawback of difficulty in controlling the forces to be applied in order to guarantee complete opening, without running the risk of opening the next reservoir, particularly if the opening means need to be actuated by inhalation.

In order to dispense the powder in a finely pulverized form, document U.S. Pat. No. 6,715,486 describes a dispersion chamber containing one or more balls that are driven in rotation by the flow of air and powder directed from the open reservoir towards the dispenser orifice. The dispersion chamber breaks up clumps of the powder in satisfactory manner, and has a positive effect on flow resistance by reducing it. However, the balls require a certain amount of energy in order to set them into motion, and it is only after this initial stage that they produce their maximum effect. In addition, the action of the inhalation flow on the balls is not uniform throughout the dispersion chamber, generally being greater at the inlet of the chamber compared with the opposite end, such that the ball(s) located remote from said inlet do not act in optimum manner. The action, and thus the effectiveness, of the balls also depends on the kind of powder to be expelled, and on the inhalation flow created by the user that may vary greatly from one use to another. This creates undesirable variability in the expulsion characteristics and performances. Documents EP 2 022 526, U.S. Pat. No. 6,328,033, and U.S. Pat. No. 7,185,648 describe other prior-art devices.

An object of the present invention is to provide a fluid dispenser device, in particular a dry-powder inhaler, that does not have the above-mentioned drawbacks.

In particular, an object of the present invention is to provide such an inhaler that is simple and inexpensive to manufacture and to assemble, that is reliable in use, guaranteeing metering accuracy and metering reproducibility on each actuation, providing an optimum yield with regard to the effectiveness of the treatment, by making it possible to dispense a substantial fraction of the dose to the zones to be treated, in particular the lungs, avoiding, in safe and effective manner, any risk of overdosing, and that is as compact as possible, while guaranteeing sealing and absolute integrity of all of the doses up to their expulsion.

Another object of the present invention is to provide such an inhaler that guarantees good metering accuracy and good metering reproducibility on each actuation, regardless of the orientation of the inhaler and/or regardless of the inhalation flow created by the user and/or regardless of the kind of powder to be expelled.

The present invention thus provides a powder inhaler device comprising: a body provided with a dispenser orifice; at least one reservoir containing a dose of powder to be dispensed; reservoir opening means for opening a reservoir on each actuation; a dispersion chamber including an outlet that is connected to said dispenser orifice, and an inlet that is connected to said opening means and that receives the dose of powder from said open reservoir, said dispersion chamber containing at least one movable element, such as a ball; said device further comprising remote drive means for remotely driving said at least one movable element, said drive means acting without contact with said at least one movable element so as to set it into motion in said dispersion chamber, said remote drive means comprising at least one magnet and/or at least two electrodes and/or at least one coil.

Advantageously, said at least one movable element is made out of, or coated with, a ferromagnetic material.

Advantageously, said remote drive means comprise at least one magnet, in particular an electro-magnet.

Advantageously, said drive means comprise two circularly-arcuate electro-magnets that are disposed around a portion of the periphery of the dispersion chamber.

Advantageously, said drive means comprise at least two electrodes that are disposed above and below said dispersion chamber.

Advantageously, said drive means comprise at least one coil creating a magnetic fields, in particular disposed in the proximity of said dispersion chamber.

Advantageously, said remote drive means are activated on inhalation.

Advantageously, the device includes at least one sensor, such as a flow sensor and/or a pressure sensor, said sensor detecting inhalation and simultaneously activating said remote drive means.

Advantageously, said flow sensor activates said remote drive means starting from the detection of an inhalation flow having at least one threshold rate, such as 5 liters per minute (L/min).

Advantageously, said remote drive means act on said at least one movable element so as to cause it to turn in said dispersion chamber at a speed of at least 3000 revolutions per minute (rpm), preferably of at least 5000 rpm.

Advantageously, the speed of movement of said at least one movable element in said dispersion chamber is independent of the inhalation rate, said remote drive means imparting to said at least one movable element, a speed that is substantially constant.

Advantageously, said dispersion chamber contains a plurality of balls, in particular six.

Advantageously, all of the balls have the same dimensions.

Advantageously, said opening means are perforator means comprising a needle that is adapted to perforate a reservoir on each actuation.

Advantageously, said opening means are controlled by the user inhaling, such that the reservoir is opened and emptied simultaneously, the powder driven by the inhalation flow passing through said dispersion chamber prior to being expelled through the dispenser orifice.

These characteristics and advantages and others of the present invention appear more clearly from the following detailed description, given by way of non-limiting example, and with reference to the accompanying drawings, and in which:

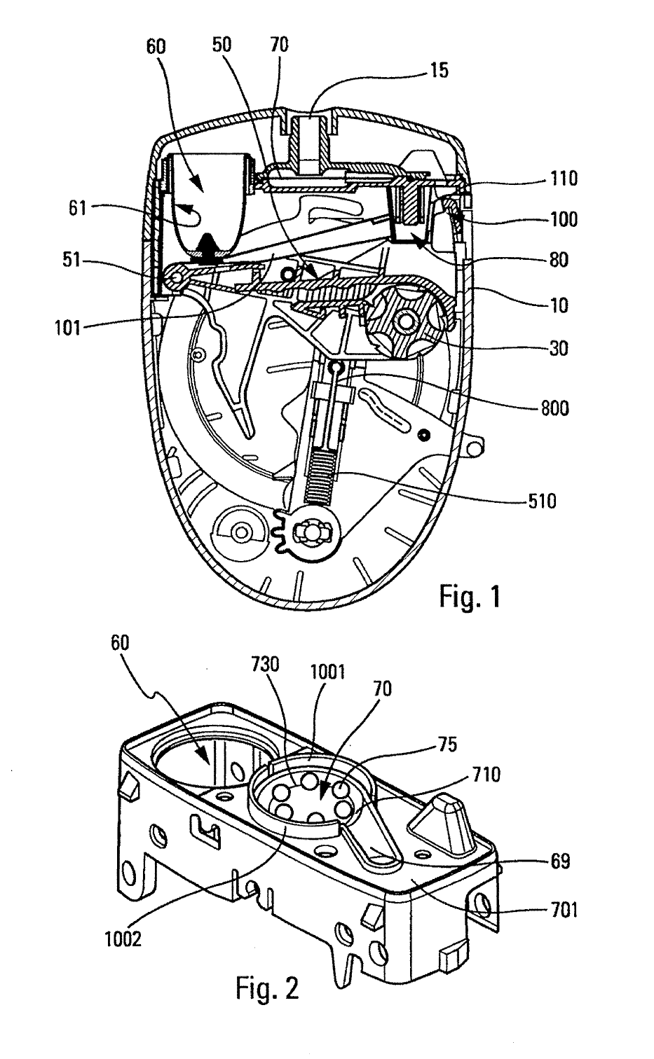

FIG. 1 is a diagrammatic section view of a powder inhaler;

FIG. 2 is a diagrammatic perspective view of a portion of the FIG. 1 inhaler device in an advantageous embodiment of the invention;

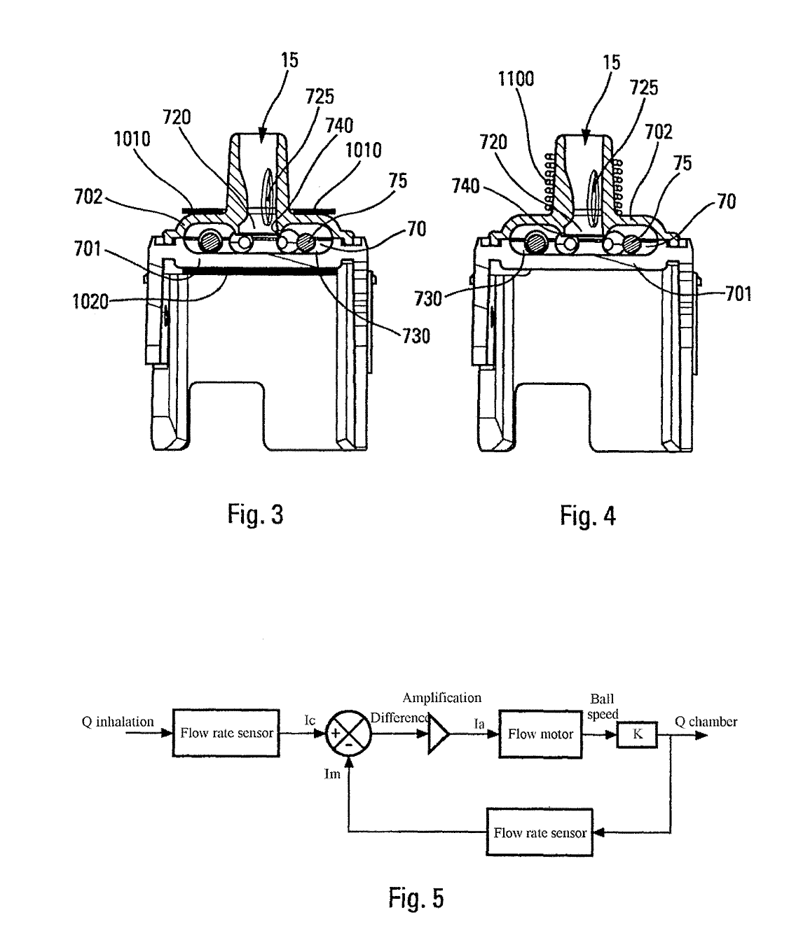

FIG. 3 is a diagrammatic side section view of a portion of the FIG. 1 inhaler device in another advantageous embodiment of the invention;

FIG. 4 is a view similar to the view in FIG. 3 in still another advantageous embodiment of the present invention; and

FIG. 5 is a servo-control diagram in a variant embodiment of the invention.

FIG. 1 shows an advantageous variant embodiment of a dry-powder inhaler. The inhaler includes a body 10 on which there can be slidably or pivotally mounted two cap-forming portions (not shown) that are adapted to be opened so as to open and pre-stress the device. The body 10 can be approximately rounded in shape, but it could be of any other appropriate shape. The body 10 includes a mouthpiece or inhaler endpiece that defines a dispenser orifice 15 through which the user inhales while the device is being actuated. The caps can be opened by pivoting about a common pivot axis, but any other opening means can be envisaged for opening the device. In a variant, the device could include a single cover instead of two.

Inside the body 10 there is provided a strip (not shown) of individual reservoirs, also known as blisters, said strip being made in the form of a flexible elongate strip on which the blisters are disposed one behind another, in manner known per se. Before first use, the blister strip can be rolled-up inside the body 10, preferably in a storage portion, and first displacement means for displacing the strip 30 are provided for progressively unrolling the blister strip and for causing it to advance. Second displacement means 50, 51 are provided for bringing a respective blister or individual reservoir into a dispensing position each time the device is actuated. The strip portion including the empty reservoirs is advantageously adapted to be rolled-up at another location of said body 10, preferably a reception portion.

The inhaler includes reservoir opening means 80 (that are shown only in very diagrammatic manner in FIG. 1) preferably comprising perforator and/or cutter means for perforating and/or cutting the closure layer of the blisters. For example, the reservoir opening means advantageously comprise a needle that is preferably stationary relative to the body 10, and against which a respective blister is displaced on each actuation by the second displacement means. The blister is thus perforated by said needle which penetrates into said blister so as to expel the powder by means of the suction of the user inhaling.

The first displacement means are adapted to cause the blister strip to advance before and/or during and/or after each actuation of the device. The second displacement means are adapted to displace the reservoir to be emptied against said perforator and/or cutter means during actuation. The second displacement means can be urged, via stressing means 800, by a resilient element 510, such as a spring or any other equivalent resilient element, said resilient element being suitable for being pre-stressed while the device is being opened. The first displacement means preferably comprise an indexer wheel 30 that receives and guides the blisters. Turning the indexer wheel causes the blister strip to advance. In a particular angular position, a given reservoir is always in a position facing the opening means. The second displacement means can include a rotary support element 50 that turns about an axis of rotation 51, said indexer wheel 30 being rotatably mounted on said support element.

An actuation cycle of the device can be as follows. While the device is being opened, the two cap-forming lateral portions are moved apart by pivoting on the body in order to open the device and thus pre-stress the device. In this position, the indexer wheel cannot be displaced towards the needle, since the second displacement means are held by appropriate blocking means 100, 110. Preferably, it is while the user is inhaling through the mouthpiece that the blocking means are unblocked, thereby causing said support element 50 to pivot and thus said indexer wheel 30 to move towards the needle, and thereby causing a reservoir to be opened.

As explained above, it is desirable for the opening means to be actuated by the user inhaling. In order to trigger the reservoir opening means by inhalation, an inhalation trigger system can be provided that advantageously comprises a unit 60 that is displaceable and/or deformable under the effect of inhalation, the unit being adapted to release the blocking means 100, 110, e.g. via a rod 101. The unit advantageously comprises a deformable air-chamber 61. Inhalation by the user causes said deformable air-chamber to deform, thereby making it possible to release said blocking means and to enable the displacement of the second displacement means, and therefore of a respective reservoir towards its opening position. The reservoir is therefore opened only on inhalation, such that it is emptied simultaneously. Thus, there is no risk of any of the dose being lost between opening the reservoir and emptying it.

In a variant, other inhalation trigger means could also be used, e.g. using a pivotable valve flap that, while the user is inhaling, pivots under the effect of the suction created by the inhalation, with pivoting of the valve flap causing the blocking means blocking the movable support means to be released, thereby causing the reservoir to be displaced towards the opening means.

The inhaler further includes a dispersion chamber 70 for receiving the dose of powder after a respective reservoir 21 has been opened. The dispersion chamber 70 is provided with at least one movable element 75, preferably made in the form of a ball. Preferably, there are six balls, as visible in FIG. 2. The balls move inside said chamber 70, so as to improve the dispensing of the air and powder mixture after a reservoir has been opened, so as to increase the effectiveness of the device.

The dispersion chamber 70 is preferably circular or elliptical in shape, with an inlet 710 that is preferably tangential in said chamber, and an outlet 720 that is perpendicular, preferably oriented along a vertical axis that passes approximately through the center of said dispersion chamber 70. Preferably, the dispersion chamber 70 is formed of two portions, a base portion 701 and a cover portion 702 that are assembled together during assembly of the device. Advantageously, the outlet 720 is formed on the cover portion 702, while the inlet 710 is formed by both portions, namely the base portion 701 and the cover portion 702. The dispersion chamber 70 includes a ball path 730 that preferably follows the shape of said dispersion chamber approximately, namely a circle or an ellipse as appropriate. The ball path 730 advantageously comprises a bottom surface that is substantially plane, and two side edge walls that are curved so as to enable the balls to move rapidly. The ball path 730 may be defined radially inside by an appropriate profile 740 that is provided on the cover portion 702, as shown in FIGS. 3 and 4. In a variant, a central projection could be provided, formed on the base portion 701 facing the outlet 720 of the dispersion chamber 70. In particular, this would be advantageous for assembly, the balls 75 positioning themselves automatically in the ball path 730 prior to fastening the cover portion 702 on the base portion 701. The outlet 720 preferably includes one or more restrictions 725 inside the channel, so as to prevent the ball(s) 75 provided in the dispersion chamber 70 from being expelled. This is a safety measure in the event of a ball 75 escaping from the ball path, e.g. during assembly. In the preferred embodiment, the dispersion chamber 70 includes a plurality of balls 75, preferably six, and the balls preferably have the same dimensions. In order to enable the balls 75 to move rapidly along the ball path 730, the width of the ball path is greater than the diameter of the balls (or greater than the diameter of the largest ball if the balls have different dimensions). Ball paths 730 can be provided that are wide enough to enable two balls to be disposed side by side in said ball path, but the ball path 730 is preferably designed to enable the passage of only one ball at a time. As visible in FIG. 2, the inlet 710 connects the dispersion chamber 70 to the perforator element 80 via a channel 69. In the variants shown, the balls 75 turn in a counter-clockwise direction, but naturally the channel 69 that leads to the inlet of the dispersion chamber could be disposed in another orientation, with the balls 75 turning in the opposite direction inside the dispersion chamber. In addition, the inlet 710 is not necessarily completely tangential, and it could even be desirable to provide an inlet 710 that is offset a little relative to the tangent.

In the invention, drive means are provided that act remotely and without contact with the ball(s) 75. An aim is to make the movement, and in particular the speed of rotation, of the balls 75 more independent of the flow rate or of the inhalation flow created by the user. On each use, the inhalation created by the user varies. However, it is desirable to have consistency in pharmaceutical performance on each use of the inhaler. The invention makes it possible to satisfy this requirement by making the movement of the balls 75 in the dispersion chamber 70 constant on each use, regardless of the rate of the inhalation flow. In addition, by sparing the inhalation flow from acting on its own to perform the task of setting the balls into motion (in addition to actuating the opening means and to expelling the powder to the dispersion chamber), the operating range of the inhaler is increased, with said inhaler thus being able to operate with lower inhalation rates. In addition, by imparting a high speed of rotation to the balls, the invention makes it possible to guarantee consistency of performance, independently of the powder to be expelled, by smoothing its powder break-up performance. By always guaranteeing the same speed of rotation of the balls, independently of the inhalation rate, and independently of the kind of powder, the invention makes it possible to optimize the reliability of the inhaler. The remote drive means of the present invention are thus different from the inhalation on its own as described in document U.S. Pat. No. 6,715,486, for example.

Advantageously, the balls 75 are made out of, or coated with, a ferromagnetic material, and the drive means may comprise one or more magnets, such as electromagnets. FIG. 2 shows two circularly-arcuate magnets 1001, 1002 that are disposed around a portion of the periphery of the dispersion chamber 70, functioning as a particle accelerator so as to impart very quickly to the balls 75, their maximum speed of rotation. Typically, the speed of rotation may be greater than 4000 rpm, advantageously at least 5000 rpm.

In a variant, as visible in FIG. 3, two electrodes 1010, 1020, that may be placed above and below the dispersion chamber 70 respectively, are used so as to act on the balls 75. FIG. 4 also shows another variant, with a coil 1100 creating a magnetic field, disposed in the proximity of the dispersion chamber 70.

Either way, the remote drive means act without contact with the balls 75 so as to set them into motion and cause them to turn in the dispersion chamber 70 by means of electromagnetic fields created during their activation. They may be powered by any appropriate power source.

Advantageously, the remote drive means 1001, 1002; 1010, 1020; 1100 are controlled by inhalation and are activated from the start of inhalation. Advantageously, for this purpose, it is possible to provide one (or more) flow and/or pressure sensor(s) (not shown), adapted to detect an inhalation flow. By way of example, the sensor may comprise a piezoelectric sensor. The sensor may advantageously be disposed inside the inhaler, e.g. in the proximity of the inhaler orifice. Starting from a threshold rate, e.g. 5 L/min, the sensor activates the drive means, and the balls 75 start very quickly to rotate. Advantageously, the balls are already turning in the dispersion chamber 70 when the powder arrives, driven by the inhalation flow after the blister has opened. Naturally, the rotation of the balls 75 may be obtained by the combined action of the remote drive means and of the inhalation flow.

FIG. 5 shows a diagram of the servo-control of the inhalation flow in an embodiment. The aim is to manage the inhalation flow rate of the user firstly by measuring said flow rate in the dispersion chamber or in the proximity thereof, then by comparing it to a setpoint, desired, or threshold flow rate that corresponds to optimum performance, and then by acting as a function of the measured difference. Q inhalation is the inhalation flow rate generated by the user. The in-line flow sensor measures the inhalation rate, the aim being to match that rate with the desired servo-control setpoint rate. Ic is a magnitude (intensity) delivered by the flow sensor, said magnitude representing an inhalation setpoint rate that is an optimum rate, making it possible to obtain good pharmaceutical performance. The comparator makes it possible to control the forward path composed of amplification and of a flow motor for generating flow to compensate for the flow lacking from the user. Amplification makes it possible to amplify the difference between the setpoint rate and the rate as actually measured, so as to be able to make use of that information. Ia is the flow rate difference to be generated by the flow motor. The flow motor represents remote drive means having action that generates the inhalation rate lacking from the user. K is experimental data, i.e. a co-efficient of proportionality between the speed of the balls and the rate in the dispersion chamber. Q chamber is the flow rate in the dispersion chamber representing the output data that it is desired to servo-control. The flow sensor of the return path is a measure of the flow rate in the dispersion chamber, and Im is the flow rate measured in the dispersion chamber.

After inhalation, when the user closes the device, all of the components return to their initial, rest position. The device is thus ready for a new utilization cycle.

The present invention therefore makes it possible to provide a dry-powder inhaler that performs the following functions:
a plurality of individual doses of powder stored in individual sealed reservoirs, e.g. 30 or 60 doses stored on a rolled-up strip;
the powder is released by perforation that is achieved by the user inhaling, the blister being perforated by means of an inhalation detector system that is coupled to a pre-stressed release system;
appropriately-shaped drive means that are engaged with blisters so as to displace the blister strip on each actuation, and to bring a new reservoir into a position in which it is to be opened by appropriate opening means;
an effective and constant dispersion of the powder prior to it being expelled, so as to limit the amount of powder that is retained, and so as to guarantee good metering accuracy and reproducibility on each actuation, even when the orientation of the inhalation is not optimum, and regardless of the inhalation rate and of the kind of powder.

Other functions are also provided by the device of the invention as described above. It should be observed that the various functions, even if they are shown as being provided simultaneously on the various embodiments of the inhaler, could be implemented separately. In particular, the inhalation trigger mechanism could be used regardless of the type of reservoir opening means, regardless of the use of a dose indicator, regardless of the way in which the individual reservoirs are arranged relative to one another, regardless of the shape of the dispersion chamber, etc. The cocking means and the inhalation trigger system could be made in some other way. The same applies for other component parts of the device.

Various modifications may also be envisaged by a person skilled in the art, without going beyond the ambit of the present invention, as defined by the accompanying claims.

1. A powder inhaler device comprising: a body provided with a dispenser orifice; at least one reservoir containing a dose of powder to be dispensed; reservoir opening means for opening a reservoir on each actuation; a dispersion chamber including an outlet that is connected to said dispenser orifice, and an inlet that is connected to said opening means and that receives the dose of powder from said open reservoir, said dispersion chamber containing at least one movable element, such as a ball; said device being characterized in that it further comprises remote drive means for remotely driving said at least one movable element, said drive means acting without contact with said at least one movable element so as to set it into motion in said dispersion chamber, said remote drive means comprising at least one magnet and/or at least two electrodes and/or at least one coil.

2. A device according to claim 1, wherein said at least one movable element is made out of, or coated with, a ferromagnetic material.

3. A device according to claim 1, wherein said remote drive means comprise at least one magnet, in particular an electro-magnet.

4. A device according to claim 3, wherein said drive means comprise two circularly-arcuate electro-magnets that are disposed around a portion of the periphery of the dispersion chamber.

5. A device according to claim 1, wherein said drive means comprise at least two electrodes that are disposed above and below said dispersion chamber.

6. A device according to claim 1, wherein said drive means comprise at least one coil creating a magnetic fields, in particular disposed in the proximity of said dispersion chamber.

7. A device according to claim 1, wherein said remote drive means are activated on inhalation.

8. A device according to claim 7, including at least one sensor, such as a flow sensor and/or a pressure sensor, said sensor detecting inhalation and simultaneously activating said remote drive means.

9. A device according to claim 7, wherein said flow sensor activates said remote drive means starting from the detection of an inhalation flow having at least one threshold rate, such as 5 L/min.

10. A device according to claim 1, wherein said remote drive means act on said at least one movable element so as to cause it to turn in said dispersion chamber at a speed of at least 3000 rpm, preferably of at least 5000 rpm.

11. A device according to claim 1, wherein the speed of movement of said at least one movable element in said dispersion chamber is independent of the inhalation rate, said remote drive means imparting to said at least one movable element, a speed that is substantially constant.

12. A device according to claim 1, wherein said dispersion chamber contains a plurality of balls, in particular six.

13. A device according to claim 12, wherein all of the balls have the same dimensions.

14. A device according to claim 1, wherein said opening means are perforator means comprising a needle that is adapted to perforate a reservoir on each actuation.

15. A device according to claim 1, wherein said opening means are controlled by the user inhaling, such that the reservoir is opened and emptied simultaneously, the powder driven by the inhalation flow passing through said dispersion chamber prior to being expelled through the dispenser orifice.
